(19) United States
(12) Patent Application Publication
(10) Pub. No.: US 2025/0157633 A1
(43) Pub. Date: May 15, 2025

(54) METHOD, DEVICE AND STORAGE MEDIUM FOR QUANTITATIVELY EVALUATING CONJUNCTIVAL CONGESTION

(71) Applicants: SHANGHAI INSTITUTE OF ENDOCRINE AND METABOLIC DISEASES, Shanghai (CN); SHANGHAI BAIYI HEALTHCARE TECHNOLOGY CO., LTD ., Shanghai (CN)

(72) Inventors: Weiqing WANG, Shanghai (CN); Guang NING, Shanghai (CN); Liyun SHEN, Shanghai (CN); Chaonan TIAN, Shanghai (CN); Dong DU, Shanghai (CN)

(21) Appl. No.: 19/019,558

(22) Filed: Jan. 14, 2025

Related U.S. Application Data
(63) Continuation of application No. PCT/CN2023/113490, filed on Aug. 17, 2023.

(30) Foreign Application Priority Data
Aug. 18, 2022 (CN) 202210992159.0

(51) Int. Cl. G16H 30/40; A61B 3/107; A61B 3/14

(52) U.S. Cl. CPC G16H 30/40; A61B 3/107; A61B 3/14

(57) ABSTRACT

A method, a device and a storage medium for quantitatively evaluating conjunctival congestion or inflammation are disclosed. The method for quantitatively evaluating conjunctival congestion or inflammation includes obtaining a video of ocular movement from the inside out or from the outside in; segmenting an eye image in the video to obtain an eye conjunctiva image, and extracting a red channel value and a blue channel value of each pixel in a conjunctival area of the eye conjunctiva image; and determining the percentage of conjunctival congestion or inflammation from the ratio of the red channel value to the blue channel value of each pixel. A precise value and/or a percentage of conjunctival congestion or inflammation can be obtained by the quantitative evaluation method.

### CROSS-REFERENCE TO RELATED APPLICATIONS

This application is a continuation of International Pat. Appl. No. PCT/CN2023/113490, filed on Aug. 17, 2023, which claims priority to Chinese Pat. Appl. No. 202210992159.0, filed on Aug. 18, 2022, the contents of each of which are incorporated by reference herein in their entireties.

### TECHNICAL FIELD

The invention relates to the technical field of eye detection, in particular to a method, a device and a storage medium for quantitatively evaluating conjunctival congestion.

### BACKGROUND DISCUSSION

The degree of conjunctival congestion or inflammation is an important reference index for doctors to evaluate the ocular inflammation of patients. The clinical doctor requires that the degree of conjunctival congestion or inflammation (light, medium or heavy) be judged manually, within 1 m of the patient. Because of the subjectivity of different doctors, there is no uniform standard for the judgment of light, medium and heavy conjunctival congestion or inflammation, which can result in misdiagnosis and missed diagnosis.

Aiming at the problem with accuracy of manual measurement as affected by human factors, the prior art proposed a shape-dependent image recognition technology scheme. As described in the Chinese patent CN111212594A (Application No.: CN201880066273.2, published on May 29, 2020), in an image captured by a camera including the eye, one or more blood vessels were identified, and the degree of conjunctival congestion is determined based on the size of the one or more blood vessels identified. In addition, in the prior art, there is another method to check the degree of conjunctival congestion by using the proportion of the red pixel value of the eye. This method specifically obtains high-resolution eye images, converts the eye images from RGB to gray, uses Gaussian filtering to find the regions with large differences in transverse and longitudinal gradients, and uses a threshold value to separate bloodshot from non-bloodshot.

However, eye inflammation is mostly conjunctival congestion or inflammation, and the congestion or inflammation in this area is mostly diffuse. Therefore, the shape-dependent recognition method used in the prior art has the following disadvantages: 1. It is easy to be affected by the depth of blood vessel color, and the red pixel values cannot be accurately extracted, resulting in inaccurate recognition. 2. Due to the characteristics and/or structure of the human eyeball, there are typically shadows at the intersection of the eyelid and the eyeball, affecting the accuracy of the method. 3. Because the eyeball is spherical, use of a single light beam to irradiate the eyeball results in uneven light.

### SUMMARY

In order to overcome the low accuracy of the prior art in measuring the degree of conjunctival congestion or inflammation, the present invention concerns a method for quantitatively evaluating conjunctival congestion or inflammation.

In order to achieve the above purpose, the present invention adopts the following technical scheme(s).

In one aspect, the present invention concerns a method for quantitatively evaluating conjunctival congestion or inflammation, including (e.g., the following steps):
Step S1: obtaining a video of ocular movement (e.g., of a patient's eye) from inside out or from outside in;
Step S2: segmenting an eye image in the video to obtain an eye conjunctiva image, and extracting a red channel value and a blue channel value of each pixel (e.g., in the conjunctival area of the image) from the eye conjunctiva image; and
Step S3: determining a percentage of conjunctival congestion or inflammation from a ratio of the red channel value to the blue channel value of each pixel.

Furthermore, one or more eye bitmaps (e.g., a “second eye bitmap”) of the eye respectively moving inward and outward to respective limit positions may be obtained in the step S2, and the eye conjunctival image may be obtained by combining a first eye bitmap of the eye moving inward to an inward limit position and a second eye bitmap of the eye moving outward to an outward limit position. In addition, the eye conjunctival image may include the lower bulbar conjunctiva only, the lower palpebral conjunctiva only, the upper bulbar conjunctiva only, the upper palpebral conjunctiva only, the lower and upper bulbar conjunctiva, the lower and upper palpebral conjunctiva, the lower bulbar and palpebral conjunctiva, the upper bulbar and palpebral conjunctiva, or the lower and upper bulbar and palpebral conjunctiva, as desired.

Furthermore, in step S3, a pixel in which the ratio of the red channel value to the blue channel value exceeds a threshold may be considered a red/bloodshot pixel. In other words, when the ratio of the red channel value to the blue channel value in one of the pixels exceeds a threshold, the one pixel is a red/bloodshot pixel. Also, the percentage of conjunctival congestion or inflammation (e.g., in the patient's eye) is the percentage of the area of the red/bloodshot pixels in the conjunctival area(s) of the eye conjunctival image.

Furthermore, the threshold may be a fixed value set in advance (e.g., a predetermined value), or a manually set value.

In another aspect, the present invention concerns a device for quantitatively evaluating conjunctival congestion or inflammation, comprising:

A video acquisition module, configured to respond to a video acquisition instruction and obtain a video of ocular movement (e.g., of a patient's eye) from inside out or from outside in:

A feature extraction module, configured to segment one or more images (e.g., of the patient's eye in the video) to obtain an eye conjunctiva image, and extract a red channel value and a blue channel value of each pixel (e.g., in a conjunctival area) in the eye conjunctiva image; and

A calculation module, configured to calculate the ratio of the red channel value to the blue channel value of each pixel to determine the percentage of conjunctival congestion or inflammation.

Furthermore, the feature extraction module may include an image processing submodule, configured to extract one or more eye bitmaps (e.g., a second eye bitmap, or first and second eye bitmaps) of the eye moving inward and outward to (or at) a respective limit position from the video.

In another aspect, the present invention concerns a computer-readable storage medium, wherein at least one program code is stored in the computer-readable storage medium, and the at least one program code is loadable and/or executable by a processor to perform a method for quantitatively evaluating conjunctival hyperplasia, congestion or inflammation as described above. The computer-readable storage medium and the processor that can load and/or execute the at least one program code may be included in the present device for quantitatively evaluating conjunctival congestion or inflammation.

Compared with the prior art, the present invention has at least the following beneficial effects:

In the present invention, the eye conjunctiva image is synthesized from eye bitmaps of the eye moving inward and outward to the limit position(s), and then the red channel value and the blue channel value are extracted from each pixel of the (synthesized) image. Since the light reflected by a bloodshot or inflamed area is mainly red, and the sclera itself has a high reflectivity for both red and blue, the red channel value is significantly higher in the bloodshot/inflamed area than the blue channel value. Where there is no bloodshot or inflammation, the red channel value and the blue channel value are equivalent, and the congestion or inflammation degree can be accurately determined by the ratio of the red channel value and the blue channel value of each pixel, so as to determine the percentage (or degree) of conjunctival congestion or inflammation.

### DESCRIPTION OF THE DRAWINGS

FIG. 1 is a flow chart of an exemplary method for quantitatively evaluating conjunctival congestion or inflammation according to the present invention;

FIG. 2 is a conceptual block diagram for the quantitative evaluation of conjunctival congestion or inflammation;

FIG. 3 is an exemplary procedure diagram for the quantitative evaluation of conjunctival congestion or inflammation of the present invention;

FIG. 4 is a structural diagram of an exemplary device for quantitatively evaluating conjunctival congestion or inflammation.

### EXAMPLES

Specific embodiments of the present invention are further described in detail in combination with the attached drawings and embodiments. The following embodiments are used to illustrate the invention, but not to limit the scope of the invention.

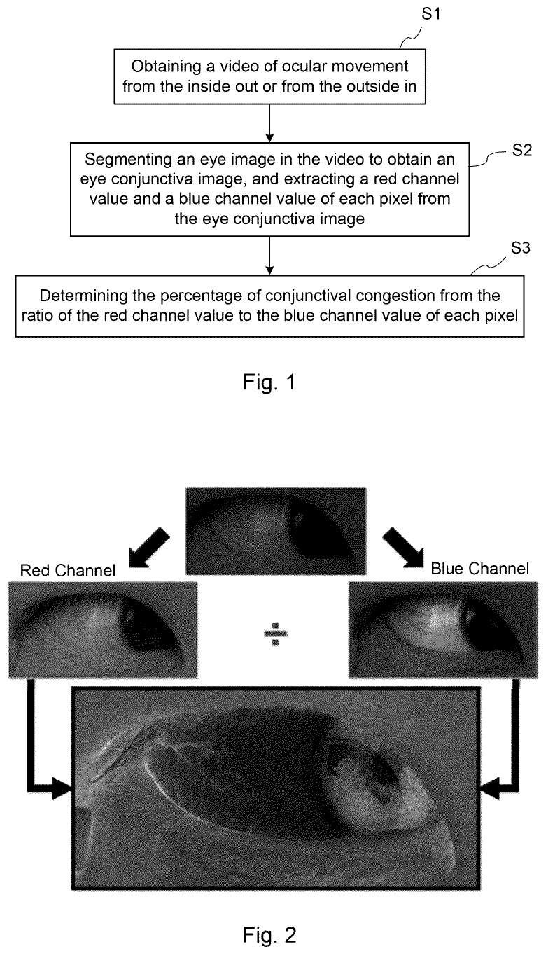

As shown in FIGS. 1-3, a method for quantitatively evaluating conjunctival congestion or inflammation includes the following steps:

Step S1: obtaining a video of ocular movement (e.g., of a patient's eye) from inside out and/or from outside in.

Specifically, in step S1, an eye evaluation or measurement device can be used to fix or set the user's eyes and head (e.g., using a head and/or chin rest or other head movement stability tool, which may be adjustable), and a camera set in front of the user's eyes (e.g., in a position that is fixed relative to the head and/or chin rest) can capture video of one of the eyes moving horizontally from inside to outside and/or from outside to inside. An indicator light (e.g., an LED, which can be included in the present device, and which may move linearly along a horizontal line) can be used to guide the user's eye movement.

Video of the ocular movements (e.g., to an inside limit position and to an outside limit position) can also be obtained through other eye devices or video capture devices.

Step S2: segmenting an eye image in the video to obtain an eye conjunctiva image, and extracting a red channel value and a blue channel value of each pixel from the eye conjunctiva image.

Specifically, in step S2, segmentation of an eye image from the video (e.g., identifying and removing from the image[s] or video one or more structures in the patient's eye not being examined, such as the iris, the pupil, the cornea, or the entire eyeball itself) can produce an eye conjunctiva image with the eye (or one or more structures therein) removed. Specifically, eye bitmaps (e.g., second eye bitmaps) with the eye moving inward and outward to respective limit positions can be extracted from the video. Then, a first extracted eye bitmap with the eye moving inward to the limit position (e.g., at the inward limit position) and a second extracted eye bitmap with the eye moving outward to the limit position (e.g., at the outward limit position) are superimposed to obtain the eye conjunctiva image without the influence of the image of the eye (or one or more structures of the eye). Then, the red channel value and blue channel value of each pixel are extracted from the eye conjunctiva image. Alternatively, the vales of the red and blue channels in each pixel of the conjunctival area in the eye conjunctiva image are determined.

It should be noted that an “eye gaze position” refers to the position of the eyeball in the eye examination, which can be divided into a first eye gaze position, a second eye gaze position, and a third eye gaze position. The first eye gaze position may refer to the eye gaze position when the patient's two eyes look straight ahead towards an infinite distance in a horizontal plane, the second eye gaze position may refer to the eye position when the eyeball moves up, down, in and/or out, and the third eye gaze position may refer to the eye position when the eyeball moves inwardly up, inwardly down, outwardly up and/or outwardly down. That is, the eye gaze position when the focal point of the eyeball moves up, under the nose, supratemporally or subtemporally may be characterized as a second or third eye gaze position. For example, the image(s) in FIG. 2 (e.g., the uppermost image) is an image of an eye in a second eye gaze position (the “in” eyeball position). Accordingly, the first, second and third eye bitmaps may refer to the images taken of the eye at the respective first, second and third eye gaze positions.

Step S3: determining a percentage (or degree) of conjunctival congestion or inflammation from a ratio of the red channel value to the blue channel value of each pixel (e.g., in the eye conjunctiva image).

In Step S3, a pixel having a ratio of the red channel value to the blue channel value that exceeds a threshold is a red/bloodshot pixel, and the percentage of conjunctival congestion or degree of inflammation is the percentage of the area of the red/bloodshot pixels in the conjunctival area of the eye conjunctiva image. Since the light reflected by the bloodshot or inflamed area (e.g., in the conjunctiva) is mainly red (see, e.g., the center left image in FIG. 2), and the sclera itself has a high reflectivity for both red light and blue light (see both images in the center row in FIG. 2), the red channel value is significantly higher in the bloodshot or inflamed area than the blue channel value, while the red channel value and the blue channel value are equivalent in the absence of a bloodshot or inflamed area (see, e.g., the lowermost image in FIG. 2). Therefore, the percentage (or degree) of conjunctival congestion or inflammation is determined by the ratio of the red channel value to the blue channel value in each pixel and, optionally, the proportion or percentage of pixels in the conjunctival are of the image that exceed the threshold. Specifically, the threshold may be a fixed value set in advance (e.g., a predetermined value). Alternatively, the threshold is a manually set value. For example, a user can enter a threshold value of 3 in the computer in advance to set pixels having a ratio of the red channel value to the blue channel value exceeding 3 as red/bloodshot. Alternatively, the program code can include a default threshold value of 3 for the red channel value: blue channel value ratio. If the RGB value of a pixel is, for example, RGB (250,245,240), the red channel value and the blue channel value are equivalent or similar, and the ratio of the two (i.e., the red channel value to the blue channel value) is 1.04 (close to 1), then the pixel is not a red/bloodshot pixel, and is likely a white pixel of the sclera itself. However, if the RGB value of a pixel is, for example, RGB (250,20,15), the ratio of the red channel value to the blue channel value is ˜17, exceeding the threshold value of 3, and the pixel is determined to be a red/bloodshot pixel. In addition to the threshold being entered in advance (or a default value), the threshold can also be a value manually set (e.g., entered into the program code) by a technician such as a doctor or operator based on his/her experience.

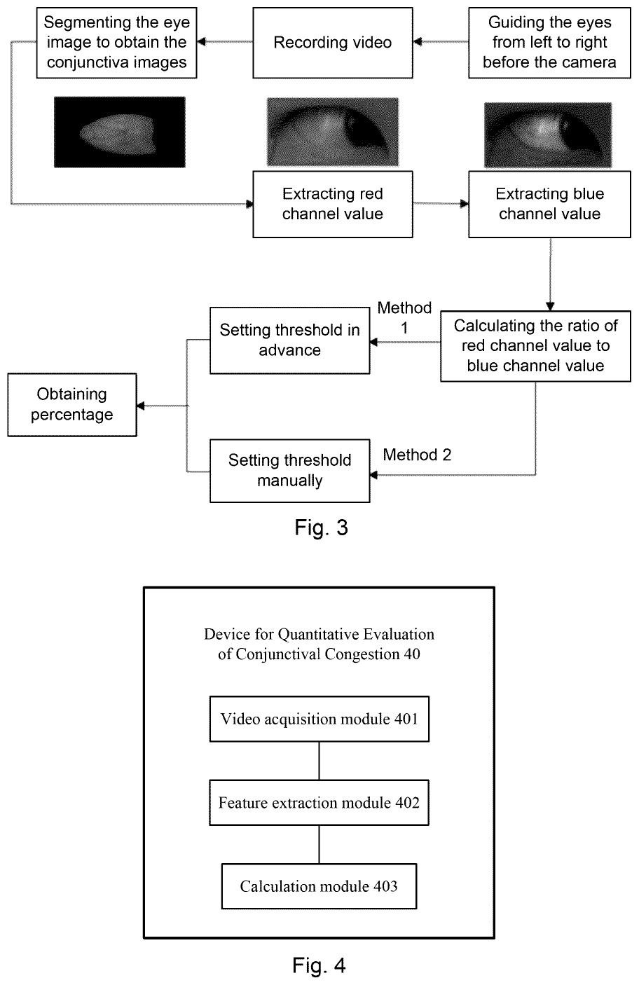

FIG. 3 more specifically shows an exemplary step diagram of the method of quantitatively evaluating conjunctival congestion or inflammation. First, the user controls the left and right (in and out) movement of the eye in front of the camera, and the video is recorded. Next, the eye image is segmented. During the segmentation of the eye image, an eye bitmap (e.g., a second eye bitmap) of the eye moving to (or at) each of the respective inward and outward limit positions (i.e., the innermost and outermost positions) is obtained. The image of the eye conjunctiva is obtained by superimposing the eye bitmap with the eye moving to or at the inward limit position and the eye bitmap with the eye moving to or at the outward limit position. Then, the red channel and blue channel values of each pixel in the eye conjunctiva image are extracted, and the ratio of the red and blue channel pixel values of each pixel (e.g., in the conjunctival area of the image) is further obtained. And according to the pre-set threshold (method 1) or manually set threshold (method 2) to determine whether each pixel is red/bloodshot, the percentage of red/bloodshot pixels is obtained. For example, a percentage of red/bloodshot pixels in the conjunctival area of the image in the range of 20-40% may be associated with irritation of the conjunctiva (e.g., from a particle or other foreign body), whereas a percentage of red/bloodshot pixels in the conjunctival area of the image of 50% or greater may be associated with infection of the conjunctiva (e.g., pink eye).

FIG. 4 is a structural diagram of a device 40 for the quantitative evaluation of conjunctival congestion or inflammation, provided by an embodiment of the present invention. The device comprises:

A video acquisition module 401, configured to respond to a video acquisition instruction and obtain a video of ocular movement from inside out or from outside in;

A feature extraction module 402, configured to segment the eye image to obtain the eye conjunctiva image, and extract the red channel value and blue channel value of each pixel from the eye conjunctiva image; and

A calculation module 403, configured to calculate the ratio of the red channel value to the blue channel value of each pixel (e.g., in the conjunctival area of the eye conjunctiva image) to determine the percentage or degree of conjunctival congestion or inflammation.

In one possible implementation, the video acquisition module 401 can control the start of the camera or other filming equipment when receiving the acquisition instruction, guide the user's eye movement by controlling a voice device or a light device, and obtain the ocular movement video. Furthermore, the feature extraction module 402 may include an image processing submodule, configured to extract one or more eye bitmaps (e.g., second eye bitmaps) of the eye moving inward and outward to the limit positions (e.g., with the pupil at the innermost and outermost positions) from the video.

In an exemplary embodiment, the present invention concerns a computer-readable storage medium, wherein at least one program code is stored in the computer-readable storage medium, and the at least one program code is loadable and/or executable by a processor (which may be in the device 40, or a separate computer or workstation in electrical communication with the device 40) to perform the method for quantitatively evaluating conjunctival hyperemia, congestion or inflammation as described above. For example, the computer-readable storage medium can comprise a read-only memory (ROM), a random access memory (RAM), a flash drive, a compact disc read-only memory (CDROM), magnetic tape, a floppy disk or an optical data storage device.

A person of ordinary skill in the art may understand that all or part of the steps to implement the above embodiments may be performed by hardware or by hardware associated with at least one program code, which may be stored in a computer-readable storage medium, such as a read-only memory, a compact disc or an optical disc.

The above are only preferred embodiments of this application and are not intended to limit the invention, and any modification, equivalent replacement, improvement, etc. made within the spirit and principles of the invention shall be included in the scope of protection of the invention.

What is claimed is:

1. A method for quantitatively evaluating conjunctival congestion or inflammation, comprising:
obtaining a video of ocular movement from inside out or from outside in;
segmenting an eye image in the video to obtain an eye conjunctiva image, and extracting a red channel value and a blue channel value of each pixel in a conjunctival area of the eye conjunctiva image; and
determining a percentage of conjunctival congestion or inflammation from a ratio of the red channel value to the blue channel value of said each pixel.

2. The method for quantitatively evaluating conjunctival congestion or inflammation of claim 1, further comprising combining a first eye bitmap of an eye moving to or at an inward limit position and a second eye bitmap of the eye moving to or at an outward limit position to obtain the eye conjunctiva image.

3. The method for quantitatively evaluating conjunctival congestion or inflammation of claim 1, wherein when the ratio of the red channel value to the blue channel value in one pixel of said each pixel exceeds a threshold, the one pixel is a red/bloodshot pixel.

4. The method for quantitatively evaluating conjunctival congestion or inflammation of claim 3, wherein a percentage of the conjunctival area of the eye conjunctiva image having the red/bloodshot pixels is a percentage of conjunctival congestion or inflammation.

5. The method for quantitatively evaluating conjunctival congestion or inflammation described of claim 3, wherein the threshold is a fixed value set in advance.

6. The method for quantitatively evaluating conjunctival congestion or inflammation described of claim 3, wherein the threshold is a manually set value.

7. A device for quantitatively evaluating conjunctival congestion or inflammation, wherein the device comprises:
a video acquisition module, configured to respond to a video acquisition instruction and obtain a video of ocular movement from inside out or from outside in;
a feature extraction module, configured to segment an eye image from the video to obtain an eye conjunctiva image, and extract a red channel value and a blue channel value of each pixel in a conjunctival area of the eye conjunctiva image; and
a calculation module, configured to calculate a ratio of the red channel value to the blue channel value of said each pixel to determine a percentage of conjunctival congestion or inflammation.

8. The device of claim 7, wherein the feature extraction module includes an image processing submodule, configured to extract a first eye bitmap of an eye moving to or at an inward limit position and a second eye bitmap of the eye moving to or at an outward limit position.

9. The device of claim 8, wherein the image processing submodule is further configured to combine the first eye bitmap and the second eye bitmap to obtain the eye conjunctiva image.

10. A computer-readable storage medium, storing at least one program code therein, the at least one program code being loadable and/or executable by a processor to perform the method for quantitatively evaluating conjunctival congestion or inflammation of claim 1.
